(19) United States
(12) Patent Application Publication
(10) Pub. No.: US 2023/0048555 A1
(43) Pub. Date: Feb. 16, 2023

(54) AEROSOL PROVISION SYSTEMS

(71) Applicant: Nicoventures Trading Limited, London (GB)

(72) Inventors: Shixiang Chen, London (GB); Ugurhan Yilmaz, London (GB); Simon Poynton, London (GB)

(21) Appl. No.: 17/791,177

(22) PCT Filed: Jan. 7, 2021
(86) PCT No.: PCT/GB2021/050034

§ 371 Date: Jul. 6, 2022

(30) Foreign Application Priority Data
Jan. 7, 2020 (GB) 2000139.2

(51) Int. Cl. A24F 40/10; A24F 40/50

(52) U.S. Cl. CPC A24F 40/10; A24F 40/50; A61M 11/042

(57) ABSTRACT

An aerosol provision device includes: a power source and control circuitry. The control circuitry is configured to cause the power source to supply electrical current in accordance with a set duty cycle to an aerosol generator so as to maintain a substantially constant average power, wherein the duty cycle is set dependent on the temperature of the aerosol generator; and wherein the control circuitry is configured to determine a voltage supplied by the power source.

### PRIORITY CLAIM

The present application is a National Phase entry of PCT Application No. PCT/GB2021/050034, filed Jan. 7, 2021, which claims priority from GB Application No. 2000139.2, filed Jan. 7, 2020, each of which is hereby fully incorporated herein by reference.

### TECHNICAL FIELD

The present disclosure relates to aerosol provision systems such as electronic smoking articles (e.g. electronic nicotine delivery systems) and the like.

### BACKGROUND

Aerosol provision systems (e.g. e-cigarettes/non-combustible tobacco heating products) generally contain an aerosolizable material, such as a reservoir of a source liquid containing a formulation, typically including nicotine, or a solid material such as a tobacco-based product, from which an aerosol is generated for inhalation by a user, for example through heat vaporization. Thus, an aerosol provision system will typically comprise an aerosol generation chamber containing a vaporizer, e.g. a heater, arranged to vaporize a portion of aerosolizable material to generate an aerosol in the aerosol generation chamber. As a user inhales on the device and electrical power is supplied to the heater, air is drawn into the device and into the aerosol generation chamber where the air mixes with the vaporized aerosolizable material and forms a condensation aerosol. There is a flow path between the aerosol generation chamber and an opening in the mouthpiece so the air drawn through the aerosol generation chamber continues along the flow path to the mouthpiece opening, carrying some of the condensation aerosol with it, and out through the mouthpiece opening for inhalation by the user.

Some aerosol provision systems include a means for controlling (e.g. limiting) the level of power supplied to a heater. For example, this can be used to help prevent adverse conditions (e.g. overheating if the aerosolizable material is running out) or to provide a desired level of aerosol formulation. Some aerosol provision systems measure an electrical resistance for the heater and use this as an indicator of temperature by taking account of how electrical resistance varies with temperature.

Approaches are described herein which seek to help provide new approaches for measuring temperature in aerosol provision systems.

### SUMMARY

In one aspect of the present disclosure there is provided an electronic aerosol provision device comprising a power source, control circuitry configured to cause the power source to supply electrical current in accordance with a set duty cycle to an aerosol generator so as to maintain a substantially constant average power, wherein the duty cycle is set dependent on the temperature of the aerosol generator; and wherein the control circuitry is configured to determine a voltage supplied by the power source.

In a further aspect of the present disclosure there is provided an electronic aerosol provision device comprising a power source; control circuitry configured to cause the power source to supply electrical current having a duty cycle to an aerosol generator so as to maintain a substantially constant average power, wherein the duty cycle is dependent on the temperature of the aerosol generator; wherein the control circuitry is configured to determine the duty cycle and to compare the duty cycle with a first duty cycle threshold dependent on a previous duty cycle.

In a further aspect of the present disclosure there is provided a control unit for use with an electronic aerosol provision device comprising: a power source; control circuitry configured to cause the power source pulses of electrical current having a duty cycle to an aerosol generator in use so as to maintain a substantially constant average power, wherein the duty cycle is dependent on the temperature of the aerosol generator; and wherein the control circuitry is configured to determine a voltage supplied by the power source.

In a further aspect of the present disclosure there is provided a control unit for use with an electronic aerosol provision device comprising: a power source; control circuitry configured to cause the power source to supply electrical current having a duty cycle to an aerosol generator in use so as to maintain a substantially constant average power, wherein the duty cycle is dependent on the temperature of the aerosol generator; wherein the control circuitry is configured to determine the duty cycle and to compare the duty cycle with a first duty cycle threshold dependent on a previous duty cycle.

In a further aspect of the present disclosure there is provided aerosol provision means comprising: power source means; control means configured to cause the power source means to supply electrical current having a duty cycle to aerosol generating means so as to maintain a substantially constant average power, wherein the duty cycle is dependent on the temperature of the aerosol generating means; and wherein the control means is configured to determine a voltage supplied by the power source means.

In a further aspect of the present disclosure there is provided aerosol provision means comprising: power source means; control means configured to cause the power source means to supply electrical current having a duty cycle to aerosol generating means so as to maintain a substantially constant average power, wherein the duty cycle is dependent on the temperature of the aerosol generating means; and wherein the control means is configured to determine the duty cycle and to compare the duty cycle with a first duty cycle threshold dependent on a previous duty cycle.

In another aspect there is provided a method of operating an aerosol provision system comprising control circuitry and a power source, wherein the control circuitry performs the method of: determining a voltage supplied by the power source; and causing the power source to supply electrical current having a duty cycle to an aerosol generator so as to maintain a substantially constant average power, wherein the duty cycle is dependent on the temperature of the aerosol generator.

In another aspect there is provided a method of operating an aerosol provision system comprising control circuitry and a power source, wherein the control circuitry performs the method of: causing the power source to supply electrical current having a duty cycle to an aerosol generator so as to maintain a substantially constant average power, wherein the duty cycle is dependent on the temperature of the aerosol generator; comparing the duty cycle with a first duty cycle threshold dependent on a previous duty cycle.

In another aspect of the present disclosure there is provided a method of operating an aerosol provision system comprising control circuitry and a power source, wherein the control circuitry performs the method of: determining a duty cycle for causing the power source to supply electrical current at a substantially constant average power to an aerosol generator, wherein the duty cycle is one of a plurality of duty cycles; and generating a probability value based on the plurality of duty cycles and a pre-determined distribution function; and comparing the probability value to a threshold value to determine if a threshold has been reached.

These and other aspects as apparent from the following description form part of the present disclosure. It is expressly noted that a description of one aspect may be combined with one or more other aspects, and the description is not to be viewed as being a set of discrete paragraphs, which cannot be combined with one another.

### BRIEF DESCRIPTION OF THE DRAWINGS

Embodiments of the invention will now be described, by way of example only, with reference to the accompanying drawings, in which:

FIG. 1 schematically represents in cross-section an aerosol provision system in accordance with certain embodiments of the disclosure;

FIG. 2 is a graph representing abnormal and normal values of PWM vs battery voltage of an aerosol provision system in accordance with certain embodiments of the disclosure;

FIG. 3 is a diagram representing voltage vs capacity for a battery of an aerosol provision system in accordance with certain embodiments of the disclosure;

FIGS. 4 and 5 schematically represent certain operating steps for aerosol provision systems in accordance with certain embodiments of the disclosure;

FIG. 6 is a flow diagram schematically representing some operating aspects of an aerosol provision system in accordance with certain embodiments of the disclosure; and

FIG. 7 shows graphs representing PWM duty cycle with respect to resistance measurements of an aerosol provision system in accordance with certain embodiments of the disclosure.

### DETAILED DESCRIPTION OF THE DRAWINGS

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to aerosol provision systems, vapor provision systems and electronic smoking systems, such as e-cigarettes and non-combustible tobacco heating products. Aerosol provision systems and vapor provision systems may include systems which are intended to generate aerosols and/or vapors from liquid source materials, solid source materials and/or semi-solid source materials, e.g. gels. Certain embodiments of the disclosure are described herein in connection with some example e-cigarette configurations (e.g. in terms of a specific overall appearance and underlying vapor generation technology). However, it will be appreciated the same principles can equally be applied for aerosol delivery systems having different overall configurations (e.g. having a different overall appearance, structure and/or vapor generation technology).

Aerosol provision systems often, though not always, comprise a modular assembly including both a reusable part (also referred to as a control unit) and a replaceable/disposable cartridge part (also referred to as a consumable part). Often the replaceable cartridge part will comprise the aerosolizable material and the vaporizer and the reusable part will comprise the power supply (e.g. rechargeable battery), activation mechanism (e.g. button or puff sensor), and control circuitry. However, it will be appreciated these different parts may also comprise further elements depending on functionality. For example, for a so-called hybrid device the cartridge part may also comprise an additional flavor element, e.g. a portion of tobacco, provided as an insert (“pod”) to add flavor to an aerosol generated elsewhere in the system. In such cases the flavor element insert may itself be removable from the disposable cartridge part so it can be replaced separately from the cartridge, for example to change flavor or because the usable lifetime of the flavor element insert is less than the usable lifetime of the aerosol generating components of the cartridge. The reusable device part will often also comprise additional components, such as a user interface for receiving user input and displaying operating status characteristics.

For modular devices, a cartridge and control unit are electrically and mechanically coupled together for use, for example using a screw thread, magnetic, latching or bayonet fixing with appropriately engaging electrical contacts. When the aerosolizable material in a cartridge is exhausted, or the user wishes to switch to a different cartridge having a different aerosolizable material, a cartridge may be removed from the control unit and a replacement cartridge attached in its place. Devices conforming to this type of two-part modular configuration may generally be referred to as two-part devices or multi-part devices.

It is relatively common for aerosol provision systems, including multi-part devices, to have a generally elongate shape and, for the sake of providing a concrete example, certain embodiments of the disclosure described herein will be taken to comprise a generally elongate multi-part device employing disposable cartridges which include an aerosolizable material and electric heater for vaporizing the aerosolizable material to form a condensation aerosol for user inhalation during use. However, it will be appreciated the underlying principles described herein may equally be adopted for different configurations of aerosol provision systems, for example single-part devices or modular devices comprising more than two parts, refillable devices and single-use disposable devices, hybrid devices which have an additional flavor element, as well as devices conforming to other overall shapes, for example based on so-called box-mod high performance devices that typically have a more box-like shape or smaller form-factor devices such as so-called pod-mod devices. More generally, it will be appreciated embodiments of the disclosure may be based on aerosol provision systems configured to incorporate the principles described herein regardless of the specific format of other aspects of such aerosol provision systems.

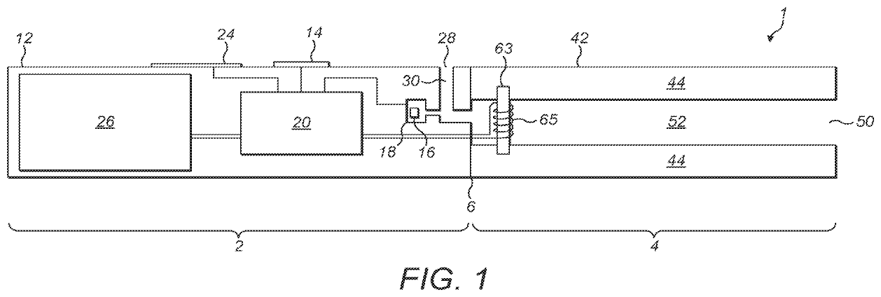

FIG. 1 is a cross-sectional view through an example aerosol provision system 1 in accordance with certain embodiments of the disclosure. The aerosol provision system 1 comprises two main components, namely a control unit 2 (which may, for example, also be referred to as a reusable part) and a consumable part 4 (which may, for example, also be referred to as a replaceable/disposable cartridge part).

In normal use the control unit 2 and the consumable part 4 are releasably coupled together at an interface 6. When the consumable part is exhausted or the user simply wishes to switch to a different consumable part, the consumable part may be removed from the control unit and a replacement consumable part attached to the control unit in its place. The interface 6 provides a structural, electrical and air path connection between the two parts and may be established in accordance with conventional techniques, for example based around a screw thread, latch mechanism, or bayonet fixing with appropriately arranged electrical contacts and openings for establishing the electrical connection and air path between the two parts as appropriate. The specific manner by which the consumable part 4 mechanically mounts to the control unit 2 is not significant to the principles described herein, but for the sake of a concrete example is assumed here to comprise a latching mechanism, for example with a portion of the cartridge being received in a corresponding receptacle in the control unit with cooperating latch engaging elements (not represented in FIG. 1). It will also be appreciated the interface 6 in some implementations may not support an electrical connection between the respective parts. For example, in some implementations a vaporizer may be provided in the control unit rather than in the consumable part.

The consumable part 4 comprises a consumable housing 42 formed of a plastics material. The consumable housing 42 supports other components of the consumable part and provides the mechanical interface 6 with the control unit 2. The consumable housing 42 in this example is generally circularly symmetric about a longitudinal axis along which the consumable part couples to the control unit 2 and has a length of around 4 cm and a diameter of around 1.5 cm. However, it will be appreciated the specific geometry, and more generally the overall shapes and materials used, may be different in different implementations.

Within the consumable housing 42 is a reservoir 44 that contains liquid aerosolizable material. The liquid aerosolizable material may be conventional, and may be referred to as e-liquid. The liquid reservoir 44 in this example has an annular shape with an outer wall defined by the consumable housing 42 and an inner wall that defines an air path 52 through the consumable part 4. The reservoir 44 is closed at each end with end walls to contain the e-liquid. The reservoir 44 may be formed in accordance with conventional techniques, for example, it may comprise a plastics material and be integrally molded with the consumable housing 42. The opening of the air path 52 at the end of the consumable part 4 provides a mouthpiece outlet 50 for the aerosol provision system through which a user inhales aerosol generated by the aerosol provision system during use.

The consumable part further comprises a wick 63 and a heater (vaporizer) 65 located towards an end of the reservoir 44 opposite to the mouthpiece outlet 50. In this example, the wick 63 extends transversely across the cartridge air path 52 with its ends extending into the reservoir 44 of e-liquid through openings in the inner wall of the reservoir 44. The openings in the inner wall of the reservoir are sized to broadly match the dimensions of the wick 63 to provide a reasonable seal against leakage from the liquid reservoir into the cartridge air path without unduly compressing the wick, which may be detrimental to its fluid transfer performance.

The wick 63 and heater 65 are arranged in the cartridge air path 52 such that a region of the cartridge air path 52 around the wick 63 and heater 65 in effect defines a vaporization region for the consumable part. E-liquid in the reservoir 44 infiltrates the wick 63 through the ends of the wick extending into the reservoir 44 and is drawn along the wick by surface tension/capillary action (i.e. wicking). The heater 65 in this example comprises an electrically resistive wire coiled around the wick 63 and is discussed further below. In this example the wick 63 comprises a glass fiber bundle, but it will be appreciated the specific wick configuration is not significant to the principles described herein. In use electrical power may be supplied to the heater 65 to vaporize an amount of e-liquid (aerosolizable material) drawn to the vicinity of the heater 65 by the wick 63. Vaporized e-liquid may then become entrained in air drawn along the cartridge air path from the vaporization region to form a condensation aerosol that exits the system through the mouthpiece outlet 50 for user inhalation. Thus electrical power can be applied to the heater 65 to selectively generate aerosol from the e-liquid in the consumable part 4. When the device is in use and generating aerosol, the amount of power supplied to the heater 65 may be varied, for example through pulse width and/or frequency modulation techniques, to control the temperature and/or rate of aerosol generation as desired.

The general configuration of the wicking element and the heating element may follow conventional techniques. For example, in some implementations, the wicking element and the heating element may comprise separate elements, e.g. a metal heating wire wound around/wrapped over a cylindrical wick, the wick, for instance, consisting of a bundle, thread or yarn of glass fibers. In other implementations, the functionality of the wicking element and the heating element may be provided by a single element. That is to say, the heating element itself may provide the wicking function. Thus, in various example implementations, the heating element/wicking element may comprise one or more of: a metal composite structure, such as porous sintered metal fiber media (Bekipor® ST) from Bekaert, a metal foam structure, e.g. of the kind available from Mitsubishi Materials; a multi-layer sintered metal wire mesh, or a folded single-layer metal wire mesh, such as from Bopp; a metal braid; or glass-fiber or carbon-fiber tissue entwined with metal wires. The “metal” may be any metallic material having an appropriate electric resistivity to be used in connection/combination with a battery. The “metal” could, for example, be a NiCr alloy (e.g. NiCr8020) or a FeCrAl alloy (e.g. “Kanthal”) or stainless steel (e.g. AISI 304 or AISI 316).

It will be appreciated the specific geometry and overall resistance of a heater in accordance with embodiments of the disclosure may be chosen having regard to the implementation at hand, for example having regard to the geometry of a wick 63 and air path 52 for an implementation of the kind shown in FIG. 1, and also the desired amount of power to be dissipated in the heater during use and the power supply voltage. For the example, the heater 65 may comprise around 10 turns of wire loosely wound around the wick 63 with an inner diameter of around 2.5 mm and that the thickness of the wire is appropriately chosen so the overall resistance of the electric heating is around 1.3 ohms. However, it will be appreciated different electric heater configuration having different electrical resistance may be used for other implementations, for example in other implementations the electric heater may have an electrical resistance within a range selected from the group comprising: 0.5 to 2 ohms, 0.8 to 1.8 ohms, 0.9 to 1.7 ohms, 1.0 to 1.6 ohms, 1.1 to 1.5 ohms and 1.2 to 1.4 ohms. Values below 0.5 Ohm could be used provided an appropriate power source is selected.

Turning now to the control unit 2, this comprises an outer housing 12 with an opening that defines an air inlet 28 for the aerosol provision system, a battery 26 for providing operating power for the aerosol provision system, control circuitry 20 for controlling and monitoring the operation of the aerosol provision system, a user input button 14, an inhalation sensor (puff detector) 16, which in this example comprises a pressure sensor located in a pressure sensor chamber 18, and a visual display 24. The control circuitry is configured to monitor the output from the inhalation sensor to determine when a user is inhaling through the mouthpiece opening 50 of the aerosol provision system so that power can be automatically supplied to the vaporizer 65 to generate aerosol in response to user inhalation. In other implementations, there may not be an inhalation sensor for detecting when a user is inhaling in the device to automatically trigger aerosol generation and instead power may be supplied to the vaporizer in response to a user manually activating the button 14/switch to trigger aerosol generation. In still other implementations, there may not be a user input button 14. In some of these implementations control circuitry 20 for controlling and monitoring the operation of the aerosol provision system may continually monitor an inhalation sensor and may activate the device in response to a determination that the user is inhaling.

The outer housing 12 may be formed, for example, from a plastics or metallic material and in this example has a circular cross-section generally conforming to the shape and size of the consumable part 4 so as to provide a smooth transition between the two parts at the interface 6. In this example, the control unit has a length of around 8 cm so the overall length of the aerosol provision system when the consumable part and control unit are coupled together is around 12 cm. However, and as already noted, it will be appreciated that the overall shape and scale of an aerosol provision system implementing an embodiment of the disclosure is not of primary significance to the principles described herein.

The air inlet 28 connects to an air path 30 through the control unit 2. The control unit air path 30 in turn connects to the cartridge air path 52 across the interface 6 when the control unit 2 and consumable part 4 are connected together. The pressure sensor chamber 18 containing the pressure sensor 16 is in fluid communication with the air path 30 in the control unit 2 (i.e. the pressure sensor chamber 18 branches off from the air path 30 in the control unit 2). Thus, when a user inhales on the mouthpiece opening 50, there is a drop in pressure in the pressure sensor chamber 18 that may be detected by the pressure sensor 16 and also air is drawn in through the air inlet 28, along the control unit air path 30, across the interface 6, through the aerosol generation region in the vicinity of the vaporizer 65 (where an aerosol generated from the aerosolizable material becomes entrained in the air flow when the vaporizer is active), along the cartridge air path 52, and out through the mouthpiece opening 50 for user inhalation.

The battery 26 in this example is rechargeable and may be of a conventional type, for example of the kind normally used in aerosol provision systems and other applications requiring provision of relatively high currents over relatively short periods. The battery 26 may be recharged through a charging connector in the control unit housing 12, for example a USB connector.

The user input button 14 in this example is a conventional mechanical button, for example comprising a spring-mounted component, which may be pressed by a user to establish an electrical contact. In this regard, the input button may be considered to provide a manual input mechanism for the aerosol provision system, but the specific manner in which the button is implemented is not significant. For example, different forms of mechanical button or touch-sensitive button (e.g. based on capacitive or optical sensing techniques) may be used in other implementations. The specific manner in which the button is implemented may, for example, be selected having regard to a desired aesthetic appearance.

The display 24 is provided to give a user a visual indication of various characteristics associated with the aerosol provision system, for example current power and/or temperature setting information, remaining battery power, and so forth. The display may be implemented in various ways. In this example, the display 24 comprises a conventional pixilated LCD screen that may be driven to display the desired information in accordance with conventional techniques. In other implementations, the display may comprise one or more discrete indicators, for example LEDs, that are arranged to display the desired information, for example through particular colors and/or flash sequences. More generally, the manner in which the display is provided and information is displayed to a user using the display is not significant to the principles described herein. Some embodiments may not include a visual display and may include other means for providing a user with information relating to operating characteristics of the aerosol provision system, for example using audio signaling or haptic feedback, or may not include any means for providing a user with information relating to operating characteristics of the aerosol provision system.

The control circuitry 20 is suitably configured/programmed to control the operation of the aerosol provision system to provide functionality in accordance with embodiments of the disclosure as described further herein, as well as for providing conventional operating functions of the aerosol provision system in line with the established techniques for controlling such devices. The control circuitry (processor circuitry) 20 may be considered to logically comprise various sub-units/circuitry elements associated with different aspects of the aerosol provision system's operation in accordance with the principles described herein and other conventional operating aspects of aerosol provision systems, such as display driving circuitry and user input detection. It will be appreciated the functionality of the control circuitry 20 can be provided in various different ways, for example using one or more suitably programmed programmable computer(s) and/or one or more suitably configured application-specific integrated circuit(s)/circuitry/chip(s) / chipset(s) configured to provide the desired functionality.

To generate an aerosol using the vapor provision system of FIG. 1, electrical power from the battery 26 is supplied to the heater 65 under control of the control circuitry 20. When the aerosol provision system is on, i.e. actively generating an aerosol, power may be supplied to the heater in a pulsed fashion, for examples using a pulse width modulation (PWM) scheme to control the level of power being delivered. Thus, the power supplied to the electric heater during a period of aerosol generation may comprise an alternating sequence of on periods during which power is connected to the electric heater and off periods during power is not connected to the electric heater. The cycle period for the pulse width modulation (i.e. the duration of a neighboring pair of an off and an on period) is in this example 0.002 s (2 ms) (i.e. the pulse width modulation frequency is 500 hertz). The proportion of each cycle period during which power is being supplied to the heater (i.e. the length of the on period) as a fraction of the cycle period is the so-called duty cycle for the pulse width modulation. In accordance with certain embodiments of the disclosure, the control circuitry of the aerosol provision system may be configured to adjust the duty cycle for the pulse width modulation to vary the power supplied to the heater, for example to achieve a target level of average power or to achieve a target temperature.

As noted above, some aerosol provision systems may include means for measuring a temperature of a heater for vaporizing aerosolizable material. Some of these aerosol provision systems may use a separate temperature sensor for measuring the temperature of the heater while others may measure an electrical resistance for the heater and use this to determine its temperature by taking account of how electrical resistance varies with temperature. One drawback of using a separate temperature sensor to measure temperature is increased structural complexity and part count. One drawback of solely relying on electrical resistance to measure temperature is low sensitivity due to the relatively low temperature coefficient of resistance associated with some materials commonly used for heaters in aerosol provision systems.

Whereas the embodiments discussed above with reference to FIG. 1 have to some extent focused on devices having a liquid aerosolizable material, as already noted the same principles may be adopted for devices based on other aerosolizable materials, for example solid materials, such as plant derived materials, such as tobacco derivative materials, or other forms of aerosolizable material, such as gel, paste or foam based aerosolizable materials. Thus, the aerosolizable material may, for example, be in the form of a solid, liquid or gel, which may or may not contain nicotine and/or flavorants. In some embodiments, the aerosolizable material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosolizable material may for example comprise from about 50 wt %, 60 wt % or 70 wt % of amorphous solid, to about 90 wt %, 95 wt % or 100 wt % of amorphous solid.

The aerosolizable material (which may also be referred to as aerosol generating material or aerosol precursor material) may in some embodiments comprise a vapor- or aerosol-generating agent or a humectant. Example such agents are glycerol, propylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

Furthermore, and as already noted, it will be appreciated the above-described approaches may be implemented in aerosol provision systems, e.g. electronic smoking articles, having a different overall construction than that represented in FIG. 1. For example, the same principles may be adopted in an aerosol provision system which does not comprise a two-part modular construction, but which instead comprises a single-part device, for example a disposable (i.e. non-rechargeable and non-refillable) device. Furthermore, in some implementations of a modular device, the arrangement of components may be different. For example, in some implementations the control unit may also comprise the vaporizer with a replaceable cartridge providing a source of aerosolizable material for the vaporizer to use to generate aerosol.

Furthermore still, in some examples the aerosol provision systems may further include a flavor insert (flavoring element), for example a receptacle (pod) for a portion of tobacco or other material, arranged in the airflow path through the device, for example downstream of the vaporizer, to impart additional flavor to aerosol generated by the vaporizer (i.e. what a hybrid type device).

As used herein, the terms “flavor” and “ flavorant”, and related terms, refer to materials which, where local regulations permit, may be used to create a desired taste or aroma in a product for adult consumers. The materials may be imitation, synthetic or natural ingredients or blends thereof. The material may be in any suitable form, for example, oil, liquid, or powder.

In accordance with certain embodiments of the disclosure, an aerosol provision device comprises a power source and a control circuitry (e.g. a controller) configured to cause the power source to supply pulses of electrical current having a duty cycle to an aerosol generator so as to maintain a substantially constant average power. The duty cycle of the pulses of electrical is dependent on the temperature of the aerosol generator. The control circuitry of the aerosol provision device is also configured to determine a voltage supplied by the power source and thus is able to advantageously use the measured voltage in conjunction with the required duty cycle to ensure safe operation of the system.

In accordance with certain embodiments of the disclosure, the control circuitry 20 of the aerosol provision system 1 may be configured to adjust the duty cycle for the pulse width modulation to ensure a target average power is supplied to the heater (e.g. a target average power may be a required power to supply an amount of energy per second to the heater). The required duty cycle to supply a particular average power to the aerosol generator may depend upon the resistance of the circuit containing the aerosol generator (e.g. a circuit containing a heater element) and the load voltage applied to the circuit. By circuit, it is meant a set of electrical components, including connections (e.g. wires), through which a current passes in response to a potential difference (i.e. voltage difference). The maximum power that can be delivered to a circuit is equal to voltage² /resistance (P=V²/R). As described above, to provide a reduced power a duty cycle can be used such that P(target)=Duty Cycle*P(max), where P(target) is the time averaged power.

The voltage applied to a circuit of the aerosol provision system 1 is generally related to the power supply voltage (e.g. battery voltage) although may be modified by various components within the system. The voltage supplied by a power source, such as a battery or a capacitor, during discharge changes dependent on the amount of charge stored in the power source. Particularly for a battery, the rate of change of the supply voltage may vary due to the characteristics of the battery (e.g. dependent on battery chemistry, the particular crystal structure and any phase transitions, which may occur during discharge). Furthermore, between different charge-discharge cycles the battery composition may permanently change, (e.g., the battery capacity degrades over time) and therefore the rate of change of the supply voltage may differ between different discharges. As a result, there is a degree of uncertainty surrounding what may be the particular voltage output at any given time during the charge-discharge cycle.

Generally speaking, the resistance of electrical conductors within the circuit is dependent on the temperature of the various electrically conductive components. In general, the resistance of an electrically conductive component increases as the temperature of the component increases. Furthermore, the resistance of a component compared to equivalent components may vary (e.g. where the aerosol generator is a heater element, the resistance of the heater element may vary by +−10%) due to the accuracy of manufacturing processes. It will be appreciated that while better machinery and improved manufacturing processes can be used to reduce the variance in heater resistance, these generally result in increased costs in the manufacturing process. Aerosol generators for use with the aerosol provision system are manufactured with resistances within an allowed tolerance (e.g. varying by no more than a set amount) with the aerosol provision system being configured to operate with any aerosol generator having a resistance within that tolerance. It will be appreciated that the operation of a device may differ significantly between when a resistor is of an optimum resistance and when the resistor is at the limit of the tolerance. For example, if the resistance is higher than optimum, then it is necessary to have a higher duty cycle to provide the same target power in comparison to an optimum resistor.

As noted above, some aerosol provision systems may rely on measurements of electrical resistance to determine temperature. However as well as having low sensitivity due to the relatively low temperature coefficient of resistance associated with some materials commonly used for heaters in aerosol provision systems, the determination of the temperature of the heater can be significantly affected by any variance in either the resistance of the heater (e.g. due to manufacturing tolerances) or the supplied voltage. Example embodiments of the disclosure instead use duty cycle as an indicator of temperature rather than resistance. By having control circuitry 20 monitoring the supply voltage the control circuitry is advantageously able to identify values of duty cycle indicative of abnormal conditions independently of the tolerance of the aerosol generator resistance and the battery voltage. Said abnormal conditions may include overheating of the aerosol generator which may be caused by a lack of liquid or other vaporizable material in contact with the aerosol generator (i.e. in normal usage the temperature of the aerosol generator is moderated by the production of aerosol). The abnormal conditions may be identified based on a higher or lower than expected duty cycle for a particular voltage and/or in comparison to an earlier value of duty cycle (e.g. a value determined in a calibration test). In some examples, the control unit may identify a value of duty cycle as abnormal if the value of duty cycle is more than a target (or expected) duty cycle for a particular voltage or range of voltages.

The term target duty cycle (e.g. first and second target duty cycles) is used to describe a value (or range of values) against which the duty cycle can be compared. The term target duty cycle is interchangeably used with the terms target duty cycle threshold and duty cycle threshold.

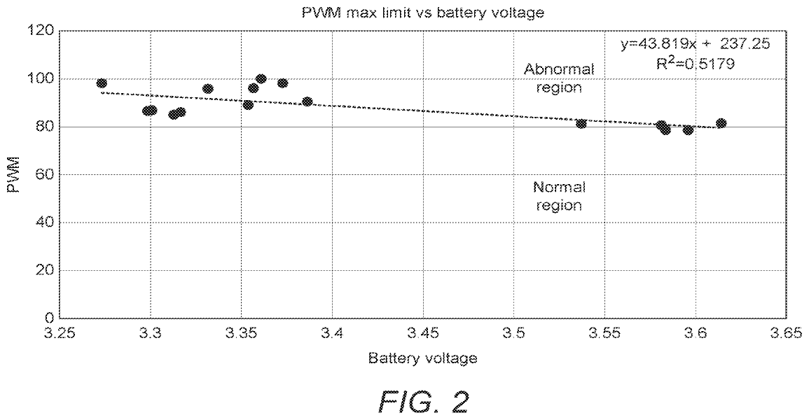

The target duty cycle may be greater than 0.85 (85%), greater than 0.90 (90%), greater than 0.95 (95%), greater than 0.98 (98%) or greater than 0.99 (99%). It will be appreciated that the duty cycle cannot exceed 1.00 (100%). The target duty cycle may be different for different voltages and/or voltage ranges thereby advantageously allowing the system to reliably indicate abnormal operation (e.g. indicative of a lack of aerosolizable material) whilst being adaptable to changes to the operating characteristics of the power source. FIG. 2 is a graph representing abnormal and normal values of PWM vs battery voltage of an aerosol provision system in accordance with certain embodiments of the disclosure. FIG. 2 depicts a plurality of data points determined as corresponding to the detection of an abnormal condition in an aerosol provision system comprising a wick, coil heater and a battery power source. The abnormal condition corresponds to the occurrence of a lack or reduced level of aerosolizable liquid at the wick. The determinations are empirically based on measurements of resistance of the coil.

For examples where the power source is a battery, a different target duty cycle can be used dependent on if the battery is close to fully charged or not close to fully charged.

For such a battery, the voltage supplied is dependent on the charge of the battery.

A threshold line separating an “abnormal region” of PWM values from a “normal region” of PWM values is also depicted in FIG. 2. The threshold line indicates the target duty cycle for a range of measured battery voltages. The threshold line is a linear fit (i.e. “Y=a*X+b”) based on the data points indicating abnormal conditions. The threshold line can be used to determine if subsequent PWM values (i.e. those taken after the threshold line has been determined) are normal or abnormal. For example, a PWM value can be compared to the threshold line and if it falls under the threshold line (i.e. has a value less than the threshold line for a particular battery voltage) then the PWM value is normal; whereas if the PWM value is above the line (i.e. has a value greater than the threshold line for a particular battery voltage) then the PWM value is abnormal. In the particular example shown in FIG. 2 the fitted rate of change of the threshold line is −43.819%/V (i.e. “a” in the formula above) and the Y-axis intercept is 237.57 (i.e. “b” in the formula above). It will be appreciated that the fitted values will be dependent on the particular aerosol provision system (e.g. type of aerosol generator, composition of liquid). In some examples, a threshold line may be fitted using a non-linear equation.

In some examples, the control circuitry 20 is configured to determine the target duty cycle based on the measured voltage delivered by the battery. In some of these examples, the control circuitry 20 may determine a target duty cycle based on the measured voltage by comparing the measured voltage to a source of comparison data (e.g. a look-up table), or by inputting the measured voltage into a formula for calculating the target duty cycle (e.g. the formula defining the threshold line of FIG. 2), and then inferring an abnormal condition as a result of the measured duty cycle being greater than the target duty cycle.

In some other examples, where the control unit is configured to identify the value of duty cycle as abnormal based on a comparison with an earlier value of duty cycle (e.g. a target duty cycle is based on the earlier value of duty cycle), the control unit may identify a value of duty cycle as abnormal if the determined duty cycle is greater than either a relative or absolute change with respect to the earlier value of duty cycle. For example the control unit may identify a value of duty cycle as abnormal if the determined duty cycle is greater than 1.05 multiplied by (e.g. 105% of) the earlier value of duty cycle, greater than 1.1 multiplied by (e.g. 110% of) the earlier value of duty cycle, greater than 1.2 multiplied by (e.g. 120% of) the earlier value of duty cycle, or greater than 1.3 multiplied by (e.g. 130% of) the earlier value of duty cycle. Alternatively, the control unit may identify a value of duty cycle as abnormal if the determined duty cycle is greater than the earlier value of duty cycle plus 0.05(+5%), greater than the earlier value of duty cycle plus 0.10 (+10%), or greater than the earlier value of duty cycle plus 0.15 (+15%). It will be appreciated that the duty cycle cannot exceed 1.00 (100%).

The target duty cycle can be different for different voltages and/or voltage ranges. For example, the target duty cycle determined based on the earlier value of duty cycle may be larger in a first voltage range than in a second voltage range (e.g. 120% of the earlier value of duty cycle in the first voltage range and 110% of the earlier value of duty cycle in the second voltage range), thereby advantageously allowing the system to reliably indicate abnormal operation (e.g. indicative of a lack of aerosolizable material) whilst being adaptable to changes to the operating characteristics of the power source. For example where the power source is a battery, a different target duty cycle can be used dependent on if the battery is close to fully charged or not close to fully charged.

Furthermore, the rate by which the voltage changes as the battery discharges may also be dependent on the charge of the battery. Typically the voltage supplied by a battery changes faster, as it discharges, when the device is either close to fully charged or close to fully discharged. The voltage supplied by the battery between these two regions tends to change slower. It will be appreciated that the exact characteristics of the change in voltage with charge (dV/dC), are dependent on the composition of the battery. When the change in voltage with charge is faster (e.g. close to fully charged), the target duty cycle can be chosen to allow greater variation in the duty cycle from an expected value. When the change in voltage with charge is slower (e.g. a mid region), the target duty cycle can be chosen to allow lesser variation in the duty cycle from an expected value. Ideally, the target duty cycle for any regime allows for the reliable detection of abnormal conditions with minimal false positives.

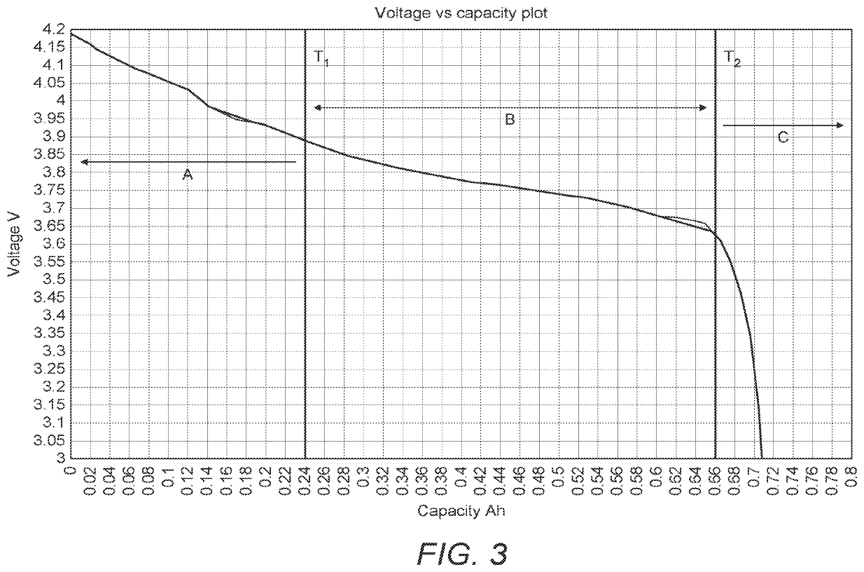

FIG. 3 is a diagram representing voltage vs capacity for a battery of an aerosol provision system in accordance with certain embodiments of the disclosure. The capacity refers to the amount of charge discharged by the battery at the time of the measurement and is defined with respect to a zero point corresponding to the battery being fully charged. The units of capacity are Amp-hours or Ah. For reference, current=charge per second (I=Q/t) and capacity=current multiplied by time which is equivalent to charge (I*t=Q). The battery voltage refers to the voltage supplied by the battery for a particular capacity (i.e. after a particular amount of charge has been discharged). In the example shown, at maximum capacity (0), the battery has an output voltage of approximately 4.18 V. In the example shown, the battery voltage falls sharply after approximately 3.63 V (in line with the vertical line marked “T₂”) which corresponds approximately with the discharge of 0.66 Ah. Generally speaking the useful capacity range of the battery is between the maximum of approximately 4.18 V and approximately 3.63 V, after which the voltage falls rapidly. Hence, for the example shown, the useful capacity of the battery may be said to be 0.66 Ah (i.e. discharge in regions A+B as shown in FIG. 3).

Beyond the value of approximately 3.63V further discharge of the battery may result in damage to the battery (i.e. discharge in region C as shown in FIG. 3). For example, defects may form compounds forming in the battery thereby reducing its overall charge capacity. Additionally, the drop in voltage may make the device unable to supply sufficient power to the aerosol generator. As stated previously, the maximum power that can be delivered to a circuit is equal to voltage²/resistance (P=V²/R). If the voltage falls too far then the voltage may not be sufficient to supply the required target power. For example, while the duty cycle can be increased to compensate for a falling voltage, the duty cycle cannot be increased passed 1.0 (i.e. the aerosol generator cannot be powered more than 100% of the time). To avoid damage and/or insufficient power being supplied, the control circuitry is configured to determine if the measured voltage is above or below the threshold value “T₂” (i.e. a second voltage threshold).

When the control circuitry determines that the measured voltage is below T₂ the control circuitry is configured to perform an action that may indicate to the user that the threshold has been reached and that the battery needs to be recharged. For example, the aerosol provision system may turn off, the aerosol provision system may cease the supply of electrical current to the aerosol generator (but may otherwise remain “on”), and/or aerosol provision system may provide an indication to the user, e.g. through a feedback mechanism such as a sound, vibration, or light feedback mechanism. In some examples, the aerosol provision system communicates (via a wired or wireless connection) with a separate device which is configured to provide a feedback mechanism to feedback to the user.

In some examples, the control circuitry 20 is also configured to determine if the measured voltage of the power supply (e.g. battery) is above or below a threshold value “T₁” (i.e. a first voltage threshold). The first voltage threshold is greater than the second voltage threshold and is a value in the usable voltage range (i.e. between 4.18 V and 3.63 V for the example shown in FIG. 3). When the supplied voltage is above T₁ the battery is in the first discharge regime “A” and when the supplied voltage is below T₁ (but above T₂) the battery is in the second discharge regime “B”.

The first discharge regime “A” corresponds to discharge when the battery is almost fully charged. As an example, the first voltage threshold may be a constant value dependent on the voltage of the power supply (e.g. battery) used when it is fully charged. The first voltage threshold “T₁” may be any value selected from the group comprising 95% of the voltage of the power source at full charge, 90% of the voltage of the power source at full charge, and 85% of the voltage of the power source at full charge. It will be appreciated that the exact voltage value will be dependent on the specific characteristics of the power supply used in the aerosol provision device.

The inventor of the present invention has noticed that a voltage during the first discharge regime “A” can vary by as much as 5% between different charge-discharge cycles. In contrast, the voltage during the second discharge regime “B” can vary by a lesser amount (for example, 2%). As previously stated, the duty cycle depends on the voltage and the resistance of the aerosol generator (and other components of the relevant circuit). If solely looking at duty cycle, any variance or error in the resistance or voltage may result in the control unit wrongly identifying the duty cycle as abnormally high since the system would not be adaptive to changes to the operation of the power source or errors resulting from manufacturing tolerances in components forming part of the circuit (e.g. the heater or the power source itself). To cope with the difference in voltage variance, and in particular in view of the higher likelihood of variance in the first region, when the measured voltage is above the first voltage threshold the control circuitry 20 is configured to compare the duty cycle to a first target duty cycle. As will be discussed in more detail below, a second target duty cycle can be used when the measured voltage is lower than the first voltage threshold. As such the target duty cycle above and below the first voltage threshold can be selected differently to provide improved anomaly detection in each of these regimes.

In some examples, the first target duty cycle is a constant value stored in memory that is readable by the control circuitry 20, where the constant value is selected, or otherwise chosen, to be a reliable comparison value for determining abnormally high duty cycles. The memory may be a memory contained in the cartridge part 4, a memory contained in the control part 2 (e.g. a memory associated with the control circuitry 20), or a memory of a separate device in wired or wireless communication with the aerosol provision system. The first target duty cycle may be written during the manufacturing process or may be written as part of a software update occurring at a later time. In some examples, the first target duty cycle is a constant value in a range selected from the group comprising greater than 0.85 (85%), greater than 0.90 (90%), greater than 0.95 (95%), and greater than 0.98 (98%).

In some examples, rather than relying on a constant value for the first target duty cycle, the control circuitry 20 is configured to determine a particular value for the first target duty cycle based on the measured voltage as this can improve the reliability of the comparison of the first target duty cycle with the duty cycle as the comparison is more specific to the particular voltage. In some examples, the control circuitry 20 is configured to firstly determine whether the measured voltage exceeds T₁ and, if it does, to secondly determine a value for the first target duty cycle based on the measured voltage before comparing the current duty cycle (i.e. to be used by the control circuitry 20 to supply a target average power to the aerosol generator) with the determined first target duty cycle. In some examples, the first target duty cycle is determined by comparing the measured voltage value with a source of comparison data, such as a look-up table, and identifying a pre-set value for the first target duty cycle corresponding to the measured voltage value. In other examples, the first target duty cycle may be determined based on the difference between the measured voltage value and one or both of the fully charged voltage value and T₁. For example, the first target duty cycle may be given a value from a range, such as 0.6 to 0.8, dependent on how close the measured voltage value is to the fully charged voltage value (e.g. if the measured voltage value is almost the fully charged voltage value then the first target duty cycle is almost 0.6) or T₁ (e.g. if the measured voltage value is almost T₁ then the first target duty cycle is almost 0.8). In still other examples, the measured voltage value may be an input into a formula, which is configured to output a value for the first target duty cycle based on the input.

In some examples, the reliability of the comparison to determine abnormally high duty cycles is improved by the control circuitry 20 being configured to determine the first target duty cycle based on a previous duty cycle, which therefore allows for a system-specific comparison measurement. For example, the previous duty cycle determined will have been dependent on the particular characteristics of the aerosol provision system (e.g. electrical resistance of components). The previous duty cycle may be a value of duty cycle determined during a previous activation of the aerosol generator (i.e. a previous aerosol generator activation event). In some examples, the previous activation event corresponds to an activation of the aerosol generator during a previous user puff. As examples, the previous user puff may be any of the immediately previous puff, the second previous puff, the third previous puff, the first puff within a preceding amount of time (e.g. the previous 5 minutes), or the first puff within a current user session (e.g. the first puff since the device switched from a “standby” to an “on” state).

As is common for aerosol provision systems, the aerosol provision system of FIG. 1 supports three basic operating states, namely an “off” state, an “on” state, and a “standby” state.

In the off state, the aerosol provision system is unable to generate aerosol (i.e. the power supply control circuitry is prevented from supplying power to the vaporizer/heater in the off state). The aerosol provision system may, for example, be placed in the off state between use sessions, for example, when the aerosol provision system might be set aside or placed in a user's pocket or bag.

In the on (or active) state, the aerosol provision system is actively generating aerosol (i.e. the power supply control circuitry is providing power to the vaporizer/heater, potentially in an on-off pulsed manner using PWM). The aerosol provision system will thus typically be in the on state when a user is in the process of inhaling aerosol from the aerosol provision system.

In the standby state the aerosol provision system is ready to generate aerosol (i.e. ready to apply power to the electric heater) in response to user activation, but is not currently doing so. The aerosol provision system will typically be in the standby state when a user initially exits the off state to begin a session of use (i.e. when a user initially turns on the aerosol provision system), or between uses during an ongoing session of use (i.e. between puffs when the user is using the aerosol provision system). It is more common for aerosol provision systems using liquid aerosolizable material to revert to the standby mode between puffs, whereas for aerosol provision systems using solid aerosolizable material may more often remain on between puffs to seek to maintain the aerosolizable material at a desired temperature during a session of use comprising a series of puffs.

In some examples, the duty cycle may be updated continually during a puff (i.e. during aerosol generator activation). This allows for a more responsive supply of power, for example, by adapting the duty cycle dependent on the currently (e.g. real-time) supplied voltage and aerosol generator resistance. In these examples, the previous duty cycle may be the last determined duty cycle for that previous activation. In some of these examples, the duty cycle may be repeatedly compared to the first target duty cycle as the duty cycle is updated during a puff to provide a responsive detection of abnormal conditions.

In some other examples, the duty cycle may be determined towards the start of a puff, at the end of the previous puff, or at an intermediate point between puffs. In some examples where the duty cycle is determined towards the start of a puff, there is an initial (pre-heat) phase of power supply where a certain amount of energy (or a certain amount of power over a certain amount of time) is supplied to the aerosol generator to bring the aerosol generator to, or close to, a required temperature. For these examples, the duty cycle is determined at the end of the initial (pre-heat) phase and is maintained for the remainder of the puff.

In some examples, the previous activation event corresponds to a test (e.g. a calibration, benchmarking or safety check) event of the heater. The test event comprises the control circuitry 20 causing power to be supplied to the aerosol generator and measurements of the resistance and the supplied voltage being recorded. This advantageously allows the first target duty cycle to be established as a system dependent value outside of a puff activation event. In some examples, the control circuitry 20 determines a duty cycle (i.e. the previous duty cycle) but does not cause power to be supplied to the aerosol generator in pulses having that duty cycle. Instead, the control circuity merely calculates the duty cycle that would be used if there was a puff activation. In some examples, the control circuity implements a test event when the aerosol provision system transitions from a first mode of operation to a second mode of operation (e.g. from an “off” state to an “on” or “standby” state). In some examples, the control circuity implements a test event after non-use of the aerosol provision system for an amount of time (e.g. after a 5 minute period of non-use).

In some examples, the control circuity implements a test event when a consumable (e.g. a cartridge part 4 containing aerosolizable material) is attached to the control part 2. In some of these examples, the control circuitry 20 implements the test event the first time a “new” cartridge part 4 is attached to the control part 2. By new it is meant that the cartridge part 4 has not been used previously with a control part 2 or that it is the first time the particular cartridge part 4 has been connected to the particular control part 2. The same target duty cycle threshold can be used for comparisons throughout the usage of that consumable.

In some of these examples, the control circuity 20 is configured to store the value of duty cycle calculated from the test event for the “new” cartridge part 4 in memory. In some examples, the control circuitry may store one or more duty cycle thresholds in the memory. The memory may be a memory contained in the cartridge part 4, a memory associated with the control part 2 (e.g. a memory associated with the control circuitry 20), or even a memory of a separate device in wired or wireless communication with the aerosol provision system. In some of these examples, the value stored in memory provides a benchmark value for use in calculating threshold values. In some other examples, the memory may be updated continuously.

In some examples, the first time a cartridge part 4 is used with a control part 2, a value of duty cycle (and/or optionally threshold values) can recorded in memory of the cartridge part 4. For subsequent uses of the cartridge part 4 by at least one different control part 2 to which the cartridge part 4 has been attached, control circuitry 20 of the at least one different control part can read the memory and use the stored value to calculate duty cycle thresholds (or read and use any stored duty cycle thresholds without requiring further calculation).

In some examples, the previous activation event corresponds either to an activation of the aerosol generator during a previous user puff or to a test activation event of the heater. For example, the control circuitry 20 may be configured to use a duty cycle from an activation corresponding to a previous puff in accordance with certain criteria (e.g. whether there has been a puff within the last 5 minutes, whether there has been a puff since the aerosol provision device was switched on, or whether there has been at least X number of puffs since the aerosol provision device was switched on). If the criteria are not met, then the control circuitry 20 is configured to implement a test event and use a duty cycle determined from that test event for determining the duty cycle threshold. Aerosol provision systems in accordance with these examples are adaptive to changing conditions in the consumable (e.g. deterioration of the aerosol generator with use) whilst also allowing a single benchmark to be used for a plurality of puffs.

In some examples, the first target duty cycle is in the range selected from the group comprising greater 105% of the previous duty cycle, greater 110% of the previous duty cycle, greater 120% of the previous duty cycle, and greater 130% of the previous duty cycle. In some examples, the first target duty cycle is in the range selected from the group comprising greater than the previous duty cycle plus 0.05, greater than the previous duty cycle plus 0.10, and greater than the previous duty cycle plus 0.15. In these examples, the first target duty cycle has a maximum value in a range selected from the group comprising 0.95, 0.98, 0.99 and 1.00.

In some examples, the control circuitry 20 is configured to compare the duty cycle to a second target duty cycle when the measured voltage is below the first voltage threshold. The second target duty cycle is more appropriate to voltages below the first voltage threshold in comparison to the first target duty cycle, which is more appropriate for values above the first voltage threshold. For example, below the first voltage threshold (e.g. regime “B” of FIG. 3) there is a lower variance in the voltage during operation of the device. As such, the determined duty cycle varies to a lesser extent below the first voltage threshold (than above the first voltage threshold) because the voltage which is used to calculate the duty cycle varies less in normal operation. A target duty cycle can therefore be selected which allows for less variation in the determined duty cycle from an expected duty cycle allowing for improved detection of abnormal conditions. In other words, above the first voltage threshold it is necessary to allow for more variation in the determined duty cycle from an expected duty cycle to prevent or limit the detection of false positives, which thereby also improves the detection of abnormal conditions.

In some examples, the second target duty cycle is a constant value stored in memory that is readable by the control circuitry 20. The memory may be a memory contained in the cartridge part 4, a memory contained in the control part 2 (e.g. a memory associated with the control circuitry 20), or a memory of a separate device in wired or wireless communication with the aerosol provision system. The second target duty cycle may be written during the manufacturing process or may be written as part of a software update occurring at a later time. In some examples, the second target duty cycle is a constant value in a range selected from the group comprising greater than 0.85, greater than 0.90, greater than 0.95, and greater than 0.98.

In examples where the first target duty cycle and the second target duty cycle are both constant values, the second target duty cycle is a value greater than the first duty cycle. In some of these examples, the second target duty cycle is at least 0.02 (2%) greater and preferably at least 0.05 (5%) greater than the value of the first target duty cycle (e.g. when the first target duty cycle equals 0.85 (85%), the second duty target cycle is at least 0.90 (90%)).

In some examples, the control circuitry 20 is configured to determine the second target duty cycle based on a previous duty cycle. The particular method of determining the second target duty cycle based on a previous duty cycle may be in accordance with any of the methods of determination described above for determining the first target duty cycle based on a previous duty cycle. However, where the first target duty cycle and the second target duty cycle are both determined in comparison to previous duty cycle value, the second target duty cycle is selected to be closer relatively to the previous duty cycle value than the first target duty cycle. For example, the first target duty cycle can be a value set at 110% or more of the previous duty cycle (e.g. to allow for greater variation in battery voltage above the first voltage threshold) while the second target duty cycle can be a value set at 105% or less of the previous duty cycle (e.g. as there is expected to be less variation in battery voltage below the first voltage threshold).

In some examples, the control circuitry 20 is configured to determine the second target duty cycle based on the measured voltage. The particular method of determining the second target duty cycle based on the measured voltage may be in accordance with any of the methods of determination described above for determining the first target duty cycle based on the measured voltage. In some examples where the first and second target duty cycles are pre-determined values, the second target duty cycle is a value greater than the first target duty cycle as the duty cycle increases as voltage drops. In some examples where the first and second target duty cycles are determined as an absolute change from a previous duty cycle, the second target duty cycle is a smaller absolute change from the previous duty cycle that the first target duty cycle. In some examples where the first and second target duty cycles are determined relative to a previous duty cycle, the second target duty cycle is a smaller relative change from the previous duty cycle than the first target duty cycle.

In some examples when the measured voltage is below the voltage threshold, the control circuitry 20 is configured to determine if there are abnormal conditions at the wick based on different electrical measurements or parameters other than duty cycle. For example, when the measured voltage is below the voltage threshold, the control circuitry 20 may be configured to determine abnormal conditions based on the resistance of the heater. In these latter examples, the lower variance in voltage below the voltage threshold may mean that resistance is a more suitable parameter to use for indicating abnormal conditions.

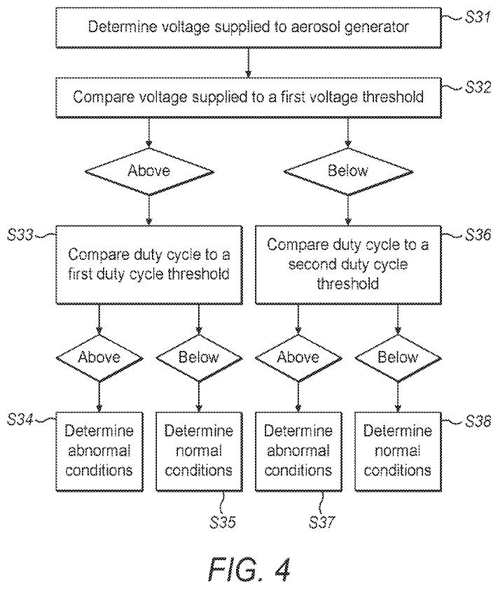

FIG. 4 is a flow diagram schematically representing some operating aspects of the aerosol provision system of FIG. 1 in accordance with certain embodiments of the disclosure.

The processing starts in step S31 in which the aerosol provision device 1 is in a “standby” state or an “on” state. Insofar as is relevant here, the processing represented in FIG. 4 is the same regardless of whether the aerosol provision system starts in the standby mode in step S31 because it has just been switched out of the off state to begin a session of use or because it is between puffs during an ongoing session of use. The manner in which the aerosol provision system is caused to switch from the off state to the standby state will be a matter of implementation and is not significant here. For example, to transition from the off state to the standby state the user may be required to press the input button 14 in a particular sequence, for example multiple presses within a predetermined time.

In S31 the control circuitry 20 is configured to determine a voltage supplied across the aerosol generator by the power source. The supplied voltage may be the load voltage or it may be the power source voltage. The voltage may be used by the control circuitry 20 to determine a duty cycle necessary to supply power at a required level and, optionally, to cause the power source to supply pulses of electrical current having a duty cycle to an aerosol generator so as to maintain a substantially constant average power.

In step S32, the control circuitry 20 compares the voltage supplied to the aerosol generator with a first voltage threshold. The comparison determines the voltage regime (e.g. A or B as shown in FIG. 3) within which the control circuitry 20 is operating and therefore the respective rules and steps to be followed. While S32 describes the control circuitry 20 comparing the voltage supplied to a single (‘first’) voltage threshold, in other examples the control circuitry 20 performs simultaneous or subsequent comparisons of the voltage supplied to other voltage thresholds. For example, the control circuitry 20 may determine if the voltage is above or below a second threshold to determine if the voltage is in a third regime (e.g. C as shown in FIG. 3). In other examples, there may be more than three regimes with each regime being separated from other regimes by a distinct voltage threshold. In each regime, the operation of the control circuitry 20 is different dependent on the respective rules and steps to be followed within that regime.

Step S33 occurs in response to the comparison of S32 determining that the supplied voltage is greater than the voltage threshold (e.g. that the supplied voltage is within the first regime A). In step S33 the control circuitry 20 compares the duty cycle to a first target duty cycle. The duty cycle for comparison is the duty cycle which the control circuitry causes or will cause to be supplied to the power source to supply pulses of electrical current to an aerosol generator at a substantially constant average power. Determination of the first target duty cycle is in accordance with any of the methods of determination described above for determining the first target duty cycle. The first target duty cycle may be determined as a preliminary step to the comparison of S33 or may be a predetermined value accessible by the control circuitry 20.

Step S34 occurs in response to the comparison of S33 determining that the duty cycle is greater than (i.e. exceeds or above) the first target duty cycle. In step S34, the control circuitry 20 determines that there are abnormal conditions. A determination of abnormal conditions may indicate that the resistance of the aerosol generator is higher than expected which can be an indication that the aerosol generator is at a higher temperature than expected. This may indicate that there is no aerosolizable material present at the aerosol generator because for some aerosol generators, such as heater-type aerosol generators, the vaporization of the aerosolizable material moderates the temperature, and in the absence of an aerosolizable material the temperature may increase beyond a normal operating temperature (which is typically the vaporization temperature of aerosolizable material).

Step S35 occurs in response to the comparison of S33 determining that the duty cycle is less than (i.e. not exceeding or below) the first target duty cycle. In step S35, the control circuitry 20 determines that there are normal conditions. A determination of normal conditions means that the aerosol generator is operating within allowed parameters. This may indicate that there is aerosolizable material present at the aerosol generation.

Step S36 occurs in response to the comparison of S32 determining that the supplied voltage is lower than the voltage threshold (e.g. that the supplied voltage is within the first regime A). In step S36 the control circuitry 20 compares the duty cycle to a second target duty cycle. The duty cycle for comparison is the duty cycle which the control circuitry causes or will cause to be supplied to the power source to supply pulses of electrical current to an aerosol generator at a substantially constant average power. Determination of the second target duty cycle is in accordance with any of the methods of determination described above for determining the second target duty cycle. The second target duty cycle may be determined as a preliminary step to the comparison of S33 or may be a predetermined value accessible by the control circuitry 20.

Step S37 occurs in response to the comparison of S36 determining that the duty cycle is greater than (i.e. exceeds or above) the first target duty cycle. In step S37, the control circuitry 20 determines that there are abnormal conditions. A determination of abnormal conditions may indicate that the resistance of the aerosol generator is higher than expected which can be an indication that the aerosol generator is at a higher temperature than expected. This may indicate that there is no aerosolizable material present at the aerosol generator because for some aerosol generators, such as heater-type aerosol generators, the vaporization of the aerosolizable material moderates the temperature, and in the absence of an aerosolizable material the temperature may increase beyond a normal operating temperature (which is typically the vaporization temperature of aerosolizable material).

Step S38 occurs in response to the comparison of S36 determining that the duty cycle is less than (i.e. not exceeding or below) the first target duty cycle. In step S38, the control circuitry 20 determines that there are normal conditions. A determination of normal conditions means that the aerosol generator is operating within allowed parameters. This may indicate that there is aerosolizable material present at the aerosol generation.

Thus, the approach of FIG. 4 represents a mode of operation in which the control circuitry 20 is configured to determine whether the aerosol generator is operating within normal or abnormal conditions. The mode of operation is adaptive to the battery level such that an appropriate duty cycle threshold is used in different regimes (i.e. ranges of battery level). A determination that the target duty cycle threshold is exceeded may therefore indicate a fault (for example a low level of liquid at the aerosol generator). The control circuitry may further be configured to control an aspect of the device based on the outcome of the process detailed in FIG. 4. For example, the control circuitry 20 may turn off the aerosol provision system, cease supplying electrical current to the aerosol generator (but may otherwise remain “on”), and/or the aerosol provision system may provide an indication to the user, e.g. through a feedback mechanism such as a sound, vibration, or light feedback mechanism.

In accordance with certain embodiments of the disclosure, an aerosol provision device comprises a power source and a control circuitry 20 configured to cause the power source to supply pulses of electrical current having a duty cycle to an aerosol generator so as to maintain a substantially constant average power. The duty cycle of the pulses of electrical current is dependent on the temperature of the aerosol generator. The control circuitry is configured to determine the duty cycle and to compare the duty cycle with a target duty cycle threshold dependent on a previous duty cycle and thus is able to advantageously react to significant changes independently of the resistance tolerance of the aerosol generator.

The target duty cycle threshold may be a pre-determined value, determined (e.g. calculated) based on a previous duty cycle and stored in memory, or may be a value that is determined (e.g. calculated) in response to the control circuitry determining a duty cycle which the control circuitry uses to cause, or potentially cause, the power source to supply pulses of electrical current to an aerosol generator at a substantially constant average power, determined based on a previous duty cycle stored in memory.

The particular method of determining the target duty cycle threshold based on a previous duty cycle may be in accordance with any of the methods of determination described above for determining the first target duty cycle or the second target duty cycle based on a previous duty cycle, and as such will not be repeated here.

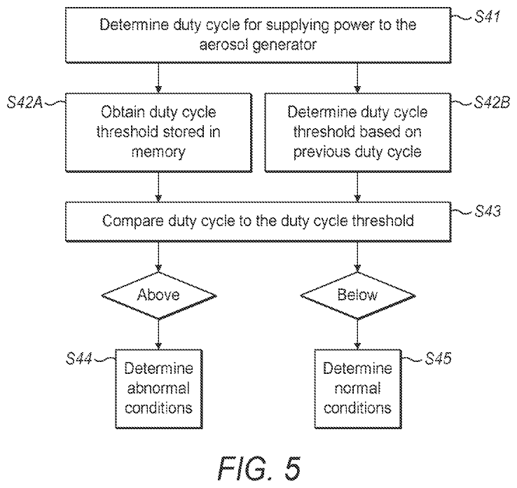

FIG. 5 is a flow diagram schematically representing some operating aspects of the aerosol provision system of FIG. 1 in accordance with certain embodiments of the disclosure.

The processing starts in step S41 in which the aerosol provision device 1 is in a “standby” state or an “on” state. Insofar as is relevant here, the processing represented in FIG. 5 is the same regardless of whether the aerosol provision system starts in the standby mode in step S41 because it has just been switched out of the off state to begin a session of use or because it is between puffs during an ongoing session of use. The manner in which the aerosol provision system is caused to switch from the off state to the standby state will be a matter of implementation and is not significant here. For example, to transition from the off state to the standby state the user may be required to press the input button 14 in a particular sequence, for example multiple presses within a predetermined time.

In S41 the control circuitry 20 is configured to determine a duty cycle for supplying power to the aerosol generator. As such, the control circuitry 20 is configured to determine a duty cycle, which the control circuitry 20 uses, or potentially uses, to cause the power source 26 to supply pulses of electrical current to an aerosol generator 65 at a substantially constant average power.

In some examples, as per step S42A, the control circuitry 20 obtains or otherwise retrieves a duty cycle threshold stored in memory. The target duty cycle threshold having been determined based on a previous duty cycle and stored in the memory prior to step S41. The memory may be a memory contained in the cartridge part 4, a memory contained in the control part 2 (e.g. a memory associated with the control circuitry 20), or a memory of a separate device in wired or wireless communication with the aerosol provision system. Aerosol provision systems 1 in accordance with these examples limits the amount of processing due after S41 is performed, as the duty cycle threshold is pre-prepared and can be used in subsequent steps.

In other examples, as per step S42B, the control circuitry 20 determines a target duty cycle threshold based on a previous duty cycle. The previous duty cycle is stored in memory. The memory may be a memory contained in the cartridge part 4, a memory contained in the control part 2 (e.g. a memory associated with the control circuitry 20), or a memory of a separate device in wired or wireless communication with the aerosol provision system. Aerosol provision systems 1 in accordance with these examples delay the determination of the threshold until it is required, thereby preventing or reducing the amount of determinations performed by the control circuitry 20. In other words, the control circuitry 20 only performs a determination if that determination will be used for subsequent steps.

While in some examples, the control circuitry 20 is configured to perform only one of either S42A or S42B; in other examples, the control circuitry is configured to perform either S42A or S42B. For example, the control circuitry 20 may determine that there is not a duty cycle threshold stored in memory, as per S42A, and instead determines a value for the duty cycle threshold, as per S42B.

In S43 the control circuitry 20 compares the duty cycle to the target duty cycle threshold. The duty cycle for comparison is the duty cycle which the control circuitry causes or will cause to be supplied to the power source to supply pulses of electrical current to an aerosol generator at a substantially constant average power. Determination of the first target duty cycle is in accordance with any of the methods of determination described above for determining the duty cycle threshold.

Step S44 occurs in response to the comparison of S43 determining that the duty cycle is greater than (i.e. exceeds or is above) the first target duty cycle. In step S34, the control circuitry 20 determines that there are abnormal conditions (i.e. abnormal operating conditions). A determination of abnormal conditions may indicate that the resistance of the aerosol generator is higher than expected which can be an indication that the aerosol generator is at a higher temperature than expected. This may indicate that there is no aerosolizable material present at the aerosol generator because for some aerosol generators, such as heater-type aerosol generators, the vaporization of the aerosolizable material moderates the temperature, and in the absence of an aerosolizable material the temperature may increase beyond a normal operating temperature (which is typically the vaporization temperature of aerosolizable material).

Step S45 occurs in response to the comparison of S43 determining that the duty cycle is less than (i.e. not exceeding or below) the first target duty cycle. In step S45, the control circuitry 20 determines that there are normal conditions (i.e. normal operating conditions). A determination of normal conditions means that the aerosol generator is operating within allowed parameters. This may indicate that there is aerosolizable material present at the aerosol generation.

Thus, the approach of FIG. 5 represents a mode of operation in which the control circuitry 20 is configured to determine whether the aerosol generator is operating within normal or abnormal conditions. The mode of operation requires the duty cycle to be compared to a value dependent on an earlier mode of operation. The mode of operation is system specific and therefore able to cope with a wider tolerance in the manufacture of the aerosol generator. A determination that the target duty cycle threshold is exceeded may therefore indicate a fault (for example a low level of liquid at the aerosol generator). The control circuitry may further be configured to control an aspect of the device based on the outcome of the process detailed in FIG. 4. For example, the control circuitry 20 may turn off the aerosol provision system, cease the supplying electrical current to the aerosol generator (but may otherwise remain “on”), and/or aerosol provision system may provide an indication to the user, e.g. through a feedback mechanism such as a sound, vibration, or light feedback mechanism.

In accordance with certain embodiments of the disclosure, with reference to FIG. 1, an aerosol provision device 1 is provided comprising control circuitry 20 and a power source (battery) 26, wherein the control circuitry 20 determines a duty cycle for causing the power source 26 to supply electrical current at a substantially constant average power to an aerosol generator, wherein the duty cycle is one of a plurality of duty cycles; and generating a probability value based on the plurality of duty cycles and a pre-determined distribution function; comparing the probability value to a threshold value to determine if a threshold has been reached.

The comparison with a threshold value may allow for a more accurate determination of anomalous conditions based on the generated probability value.

In some examples, the control circuitry 20 of the aerosol provision system 1 is configured to adjust the duty cycle for the pulse width modulation to ensure a target average power is supplied to the heater (e.g. a target average power may be a required power to supply an amount of energy per second to the heater). The required duty cycle to supply a particular average power to the aerosol generator may depend upon the resistance of the circuit containing the aerosol generator (e.g. a circuit containing a heater element) and the load voltage applied to the circuit. By circuit, it is meant a set of electrical components, including connections (e.g. wires), through which a current passes in response to a potential difference (i.e. voltage difference). The maximum power that can be delivered to a circuit is equal to voltage² /resistance (P=V²/R). As described above, to provide a reduced power a duty cycle can be used such that P(target)=Duty Cycle*P(max), where P(target) is the time averaged power.

The voltage applied to a circuit of the aerosol provision system 1 is generally related to the power supply voltage (e.g. battery voltage) although may be modified by various components within the system (e.g. a DC to DC converter). The voltage supplied by a power source, such as a battery or a capacitor, varies during discharge dependent on the amount of charge stored in the power source. Particularly for a battery, the rate of change of the supply voltage may vary due to the characteristics of the battery (e.g. dependent on battery chemistry, the particular crystal structure and any phase transitions, which may occur during discharge). Furthermore, between different charge-discharge cycles the battery composition may permanently change (e.g. the battery capacity degrades over time) and therefore the rate of change of the supply voltage may differ between different discharges. As a result, there is a degree of uncertainty surrounding what may be the particular voltage output at any given time during the charge-discharge cycle.

Generally speaking, the resistance of electrical conductors within the circuit is dependent on the temperature of the various electrically conductive components. In general, the resistance of an electrically conductive component increases as the temperature of the component increases. Furthermore, the resistance of a component compared to equivalent components may vary (e.g. where the aerosol generator is a heater element, the resistance of the heater element may vary by +−10%) due to the accuracy of manufacturing processes. It will be appreciated that while better machinery and improved manufacturing processes can be used to reduce the variance in heater resistance, these generally result in increased costs in the manufacturing process. Aerosol generators for use with the aerosol provision system are manufactured with resistances within an allowed tolerance (e.g. varying by no more than a set amount) with the aerosol provision system being configured to operate with any aerosol generator having a resistance within that tolerance. It will be appreciated that the operation of a device may differ significantly between when a resistor is of an optimum resistance and when the resistor is at the limit of the tolerance. For example, if the resistance is higher than optimum, then it is necessary to have a higher duty cycle to provide the same target power in comparison to an optimum resistor.

As previously stated, the control circuitry 20 determines a duty cycle for causing the power source to supply electrical current at a substantially constant average power to an aerosol generator. The determined duty cycle can be considered one of a plurality of determined duty cycles. The others of the plurality of determined duty cycles are previously determined duty cycles. In some examples, the previously determined duty cycles may be duty cycles determined during respective previous puff events (i.e. previous activations of the aerosol generator to generate aerosols for inhalation). In some examples, the duty cycle may be determined towards the start of a puff, at the end of the previous puff, or at an intermediate point between puffs (e.g. a set time after the puff has ended such as any time between 0.5 and 2 seconds).

In some examples where the duty cycle is determined towards the start of a puff, there may be an initial (pre-heat) phase of power supply where a certain amount of energy (or a certain amount of power over a certain amount of time) is supplied to the aerosol generator to bring the aerosol generator to, or close to, a required temperature. For these examples, the duty cycle is determined at the end of the initial (pre-heat) phase and is maintained for the remainder of the puff.

The plurality of determined duty cycles can be held in memory associated with the aerosol provision device 1 such that the control circuitry 20 is able to read and write data to the memory. In some examples, the control circuitry 20 comprises the memory. In some examples, the cartridge part 4 comprises the memory and the control circuitry 20 is configured to communicate with the memory through one or more connections. In some examples an external device (e.g. a smart phone or a server) comprises the memory and the control circuitry 20 is configured to communicate wireles sly with the external device (e.g. via a wireless transceiver). In some examples, the plurality of duty cycles is stored in multiple locations. For example, the plurality of duty cycles can be stored both within a memory of the control circuitry 20 and within a memory of the cartridge part 4. By maintaining (at least one copy of) the plurality of duty cycles outside of the control circuitry 20; if the cartridge part 4 is used with a different aerosol provision device then the different aerosol provision device can be configured to be able to access the plurality of duty cycles and therefore can perform future operations using the plurality of duty cycles.

In examples, the control circuitry 20 is configured to generate a probability value based on the plurality of duty cycles and a pre-determined distribution function. By a distribution function, it is meant a mathematical function which can be used to provide a probability value associated with a relationship of a value to a series of values, and as such, it takes its normal meaning. For example, for a cumulative distribution function the probability value is the probability that the distribution function defining the series has a value equal to or less than the value, while for a probability distribution function the probability value is the probability that a value is part of the series defined by the distribution function.

In examples, the control circuitry 20 is configured to compare the probability value to a threshold value to determine if a threshold has been reached. By comparison, it is meant that the determination is made of whether the probability value has reached the threshold value. In some examples, a probability value may have reached a threshold value when the probability value is greater than or equal to the probability value. In some examples, a probability value may have reached a threshold value when the probability value is less than or equal to the probability value.

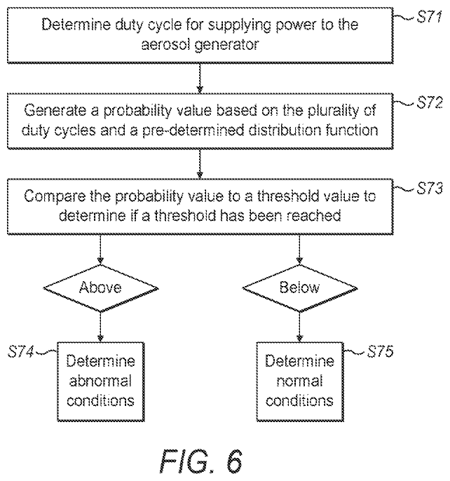

FIG. 6 is a flow diagram schematically representing some operating aspects of the aerosol provision system of FIG. 1 in accordance with certain embodiments of the disclosure.

The processing starts in step S71 in which the aerosol provision device 1 is in a “standby” state or an “on” state. Insofar as is relevant here, the processing represented in FIG. 6 is the same regardless of whether the aerosol provision system starts in the standby mode in step S71 because it has just been switched out of the off state to begin a session of use or because it is between puffs during an ongoing session of use. The manner in which the aerosol provision system is caused to switch from the off state to the standby state will be a matter of implementation and is not significant here. For example, to transition from the off state to the standby state the user may be required to press the input button 14 in a particular sequence, for example multiple presses within a predetermined time.

In S71 the control circuitry 20 determines a duty cycle for causing the power source to supply electrical current at a substantially constant average power to an aerosol generator. In some examples, the duty cycle may be determined based on either a load voltage across the aerosol generator or the power source voltage.

In step S72, the control circuitry 20 generates a probability value based on the plurality of duty cycles and a pre-determined distribution function. The probability value is generated as an output of a distribution function, where the distribution function is considered pre-determined in that the control circuitry 20 is configured to perform a mathematical operation corresponding to the distribution function. The pre-determined distribution function takes as inputs at least values related to or generated from the plurality of duty cycles. In some examples, the values may be a standard deviation and/or a mean of the plurality of duty cycles.

In some examples, the pre-determined distribution function is a cumulative log normal distribution function. Generating a probability value based on the plurality of duty cycles and a distribution function may comprise calculating, for each of the plurality of duty cycles, a respective one of a plurality of natural logarithms. Next, a mean of the plurality of natural logarithms may be calculated. Next, a standard deviation of the plurality of natural logarithms may be calculated. Next, a probability value may be generated by inputting the duty cycle, the mean and the standard deviation into the cumulative log normal distribution function, wherein the cumulative log normal distribution function provides the probability value as an output. In this example, the threshold value may be in the range 0.93 to 0.99.

In step S73, the control circuitry 20 compares the probability value to a threshold value to determine if a threshold has been reached. The comparison allows the control circuitry 20 to determine the regime within which the aerosol generator (e.g. vaporizer 65) is operating and therefore the respective rules and steps to be followed. In one example, the threshold value is in the range 0.70 to 0.85

In step S74, a determination is made that the aerosol generator is operating in an abnormal regime if the threshold has been reached. A determination of abnormal conditions may indicate that the amount or level of aerosolizable material present at the aerosol generator has fallen below a threshold.

In step S75, if the threshold has not been reached, the control circuitry 20 determines that the aerosol generator is operating in a normal regime.

In one example, the threshold is considered to have been reached if the probability value is higher than the threshold value.

In one example, the method of FIG. 6 may further comprise controlling an aspect of the aerosol provision system based on the comparison of the probability value to a threshold value. For example, controlling the aspect may comprise preventing the power source from supplying electrical current to the aerosol generator if the threshold has been reached. Alternatively, or in addition, controlling the aspect may comprise causing the power source from supplying electrical current to the aerosol generator if the threshold has not been reached. Alternatively, or in addition, controlling the aspect comprises indicating to the user that the threshold has been reached if the threshold has been reached.

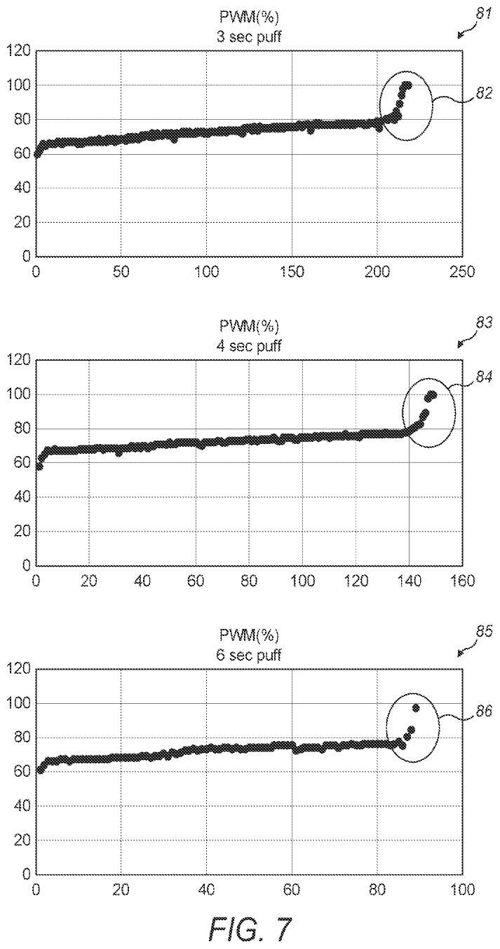

FIG. 7 shows graphs representing PWM duty cycle vs resistance measurements of an aerosol provision system in accordance with certain embodiments of the disclosure.

FIG. 7 shows a graph 81 representing correlations between a PWM duty cycle and resistance measurements of the aerosol provision system, for a puff duration of 3 seconds, where the resistance measurements may be indicative of dry out conditions. The graph 81 may relate to a specific liquid base (e.g. ice mint). The region 82 is representative of a dry-out region for the puff duration of 3 seconds.

FIG. 7 further shows a graph 83, similar to the graph 81, for a puff duration of 4 seconds. The graph 83 may relate to the same liquid base as that of graph 81. The region 84 is representative of a dry out region for the puff duration of 4 seconds.

FIG. 7 further shows a graph 85, similar to the graphs 81 and 83, for a puff duration of 6 seconds. The graph 85 may relate to the same liquid base as that of graphs 81 and 83. The region 86 is representative of a dry out region for the puff duration of 6 seconds.

In one example, for some aerosol generators, such as heater-type aerosol generators, the vaporization of the aerosolizable material moderates the temperature, and in the absence of an aerosolizable material, the temperature may increase beyond a normal operating temperature (which is typically the vaporization temperature of aerosolizable material). An absence of aerosolizable material may be caused by aerosolization of the aerosolizable material or by a reduced supply of aerosolizable materials (for example, if the flow of a liquid aerosolizable material diminishes then the resupply of aerosolizable material to the aerosol generator will reduce).

A determination of normal conditions means that the aerosol generator is operating within allowed parameters. This may indicate that there is a suitable amount of aerosolizable material present at the aerosol generation.

Thus, the approach of FIG. 6 represents a mode of operation in which the control circuitry 20 is configured to determine whether the aerosol generator is operating within normal or abnormal conditions based on a comparison between a probability value and a threshold value.

The control circuitry may further be configured to control an aspect of the device based on the outcome of the process detailed in FIG. 6. For example, the control circuitry 20 may turn off the aerosol provision system, cease supplying electrical current to the aerosol generator (but may otherwise remain “on”), and/or the aerosol provision system may provide an indication to the user, e.g. through a feedback mechanism such as a sound, vibration, or light feedback mechanism. In some examples, an indication may instruct the user to change a source of aerosolizable material (e.g. the cartridge part 4).

As noted above, some aerosol provision systems may rely on measurements of electrical resistance to determine temperature. However as well as having low sensitivity due to the relatively low temperature coefficient of resistance associated with some materials commonly used for heaters in aerosol provision systems, the determination of the temperature of the heater can be significantly affected by any variance in the resistance of the heater (e.g. due to manufacturing tolerances). In contrast, the present method allows for statistical based detection that is dependent on previously determined duty cycles (e.g. the previously determined duty cycles may be used to generate inputs for the distribution function) which are dependent on the resistance of the heater (or other aerosol generator). As such, embodiments of the disclosure provide a method of determining abnormal conditions that is not affected by the variance of the heater resistance.

Thus there has been described an aerosol provision system comprising: a power source, control circuitry configured to cause the power source to supply electrical current in accordance with a set duty cycle to an aerosol generator so as to maintain a substantially constant average power. Wherein the duty cycle is set dependent on the temperature of the aerosol generator, and wherein the control circuitry is configured to determine a voltage supplied by the power source.

Thus there has also been described an electronic aerosol provision device comprising a power source and control circuitry configured to cause the power source to supply electrical current having a duty cycle to an aerosol generator so as to maintain a substantially constant average power. Wherein the duty cycle is dependent on the temperature of the aerosol generator, and wherein the control circuitry is configured to determine the duty cycle and to compare the duty cycle with a first duty cycle threshold dependent on a previous duty cycle.

Thus there has also been described a control unit for use with an electronic aerosol provision device comprising: a power source and control circuitry configured to cause the power source pulses of electrical current having a duty cycle to an aerosol generator in use so as to maintain a substantially constant average power. Wherein the duty cycle is dependent on the temperature of the aerosol generator, and wherein the control circuitry is configured to determine a voltage supplied by the power source.

Thus there has also been described a control unit for use with an electronic aerosol provision device comprising: a power source and control circuitry configured to cause the power source to supply electrical current having a duty cycle to an aerosol generator in use so as to maintain a substantially constant average power. Wherein the duty cycle is dependent on the temperature of the aerosol generator and wherein the control circuitry is configured to determine the duty cycle and to compare the duty cycle with a first duty cycle threshold dependent on a previous duty cycle.

Thus, there has also been described aerosol provision means comprising: power source means and control means configured to cause the power source means to supply electrical current having a duty cycle to aerosol generating means so as to maintain a substantially constant average power. Wherein the duty cycle is dependent on the temperature of the aerosol generating means, and wherein the control means is configured to determine a voltage supplied by the power source means.

Thus, there has also been described aerosol provision means comprising: power source means and control means configured to cause the power source means to supply electrical current having a duty cycle to aerosol generating means so as to maintain a substantially constant average power. Wherein the duty cycle is dependent on the temperature of the aerosol generating means, and wherein the control means is configured to determine the duty cycle and to compare the duty cycle with a first duty cycle threshold dependent on a previous duty cycle.

Thus there has also been described a method of operating an aerosol provision system comprising control circuitry and a power source, wherein the control circuitry performs the method of: determining a voltage supplied by the power source; and causing the power source to supply electrical current having a duty cycle to an aerosol generator so as to maintain a substantially constant average power, wherein the duty cycle is dependent on the temperature of the aerosol generator.

Thus there has also been described a method of operating an aerosol provision system comprising control circuitry and a power source, wherein the control circuitry performs the method of: causing the power source to supply electrical current having a duty cycle to an aerosol generator so as to maintain a substantially constant average power, wherein the duty cycle is dependent on the temperature of the aerosol generator; comparing the duty cycle with a first duty cycle threshold dependent on a previous duty cycle.

Thus, there has also been described a method of operating an aerosol provision system in accordance with the following numbered clauses:

Clause 1: A method of operating an aerosol provision system comprising control circuitry and a power source, wherein the control circuitry performs the method of: determining a duty cycle for causing the power source to supply electrical current at a substantially constant average power to an aerosol generator, wherein the duty cycle is one of a plurality of duty cycles; generating a probability value based on the plurality of duty cycles and a pre-determined distribution function; and comparing the probability value to a threshold value to determine if a threshold has been reached.

Clause 2: The method of clause 1, wherein the pre-determined distribution function is an inverse log normal distribution function, and generating a probability value based on the plurality of duty cycles and a distribution function comprises: calculating, for each of the plurality of duty cycles, a respective one of a plurality of natural logarithms; calculating a mean of the plurality of natural logarithms; calculating a standard deviation of the plurality of natural logarithms; generating the probability value by inputting the duty cycle, the mean and the standard deviation into the inverse log normal distribution function, wherein the inverse log normal distribution function provides the probability value as an output.

Clause 3: The method of clause 2, wherein the threshold value is in the range 0.70 to 0.85.

Clause 4: The method of clause 1, wherein the pre-determined distribution function is an cumulative log normal distribution function, and generating a probability value based on the plurality of duty cycles and a distribution function comprises: calculating, for each of the plurality of duty cycles, a respective one of a plurality of natural logarithms; calculating a mean of the plurality of natural logarithms; calculating a standard deviation of the plurality of natural logarithms; generating the probability value by inputting the duty cycle, the mean and the standard deviation into the cumulative log normal distribution function, wherein the cumulative log normal distribution function provides the probability value as an output.

Clause 5: The method of clause 4, wherein the threshold value is in the range 0.93 to 0.99.

Clause 6: The method of any of clauses 1 to 5, wherein the threshold has been reached if the probability value is higher than the threshold value.

Clause 7: The method of any of clauses 1 to 6, wherein the method further comprises controlling an aspect of the aerosol provision system based on the comparison of the probability value to a threshold value.

Clause 8: The method of clause 7, wherein controlling the aspect comprises preventing the power source from supplying electrical current to the aerosol generator if the threshold has been reached.

Clause 9: The method of clause 7 or clause 8, wherein controlling the aspect comprises causing the power source from supplying electrical current to the aerosol generator if the threshold has not been reached.

Clause 10: The method of any one of clauses 7 to 9, wherein controlling the aspect comprises indicating to the user that the threshold has been reached if the threshold has been reached.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future.

1. An aerosol provision device comprising:
a power source;
control circuitry configured to cause the power source to supply electrical current in accordance with a set duty cycle to an aerosol generator so as to maintain a substantially constant average power, wherein the duty cycle is set dependent on the temperature of the aerosol generator; and
wherein the control circuitry is configured to determine a voltage supplied by the power source.

2. The aerosol provision system of claim 1, wherein the control circuitry is configured to determine a first duty cycle threshold based on the determined voltage.

3. The aerosol provision system of claim 2, wherein the determined voltage is compared to a source of comparison data to determine the first duty cycle threshold.

4. The aerosol provision device of claim 1,
wherein the control circuitry is configured to ascertain if the determined voltage is above or below a voltage threshold;
wherein when the determined voltage is above the voltage threshold the control circuitry is configured to compare the duty cycle to the first duty cycle threshold; and/or
wherein when the determined voltage is below the voltage threshold the control circuitry is configured to compare the duty cycle to a second duty cycle threshold.

5. The aerosol provision system of claim 2, wherein the control circuitry is configured to determine the first duty cycle threshold based on a previous duty cycle.

6. The aerosol provision system of claim 5, wherein the previous duty cycle having been determined during a previous aerosol generator activation event corresponding to a user puff.

7. The aerosol provision system of claim 5, wherein the previous duty cycle having been determined during a previous aerosol generator activation event corresponding to a test event.

8. The aerosol provision system of claim 7, wherein the test event is caused by transitioning the aerosol provision device from a first mode of operation to a second mode of operation.

9. The aerosol provision system of claim 5, wherein the first duty cycle threshold is in the range selected from the group comprising greater 105% of the previous duty cycle, greater 110% of the previous duty cycle, greater 120% of the previous duty cycle, and greater 130% of the previous duty cycle.

10. The aerosol provision system of claim 5, wherein the first duty cycle threshold is in the range selected from the group comprising greater than the previous duty cycle plus 0.05, greater than the previous duty cycle plus 0.10, and greater than the previous duty cycle plus 0.15.

11. The aerosol provision system of claim 2, wherein the first duty cycle threshold has a maximum value in a range selected from the group comprising 0.95, 0.98, 0.99 and 1.00.

12. The aerosol provision system of claim 2, wherein the second duty cycle threshold is a constant value in a range selected from the group comprising greater than 0.85, greater than 0.90, greater than 0.95, and greater than 0.98.

13. The aerosol provision system of claim 2, wherein the voltage threshold is a constant value selected from the group comprising 95% of the voltage of the power source at full charge, 90% of the voltage of the power source at full charge, and 85% of the voltage of the power source at full charge.

14. The aerosol provision system of claim 2, wherein the control circuitry is configured to determine if the measured voltage is above or below a second voltage threshold, wherein when the measured voltage is below the second voltage threshold the control circuitry is configured to perform any selected from the group comprising turning off the device, ceasing the supply pulses of electrical current having a duty cycle to the aerosol generator and provide an indication to the user, and wherein the second voltage threshold value is less than the voltage threshold.

15. An aerosol provision device comprising:
a power source;
control circuitry configured to cause the power source to supply electrical current having a duty cycle to an aerosol generator so as to maintain a substantially constant average power, wherein the duty cycle is dependent on the temperature of the aerosol generator;
wherein the control circuitry is configured to determine the duty cycle and to compare the duty cycle with a first duty cycle threshold dependent on a previous duty cycle.

16. The aerosol provision device of claim 15, wherein the first duty cycle threshold is determined as a percentage of the previous duty cycle.

17. The aerosol provision device of claim 15, wherein the control circuitry is configured to determine a voltage supplied by the power source, and wherein the first duty cycle threshold is dependent on the determined voltage.

18. The aerosol provision device of any of claim 17, wherein the first duty cycle threshold is dependent is determined as a percentage of the previous duty cycle, wherein the percentage is proportional to the determined voltage.

19. The aerosol provision device of claim 16, wherein the first duty cycle threshold is in the range selected from the group comprising greater 105% of the previous duty cycle, greater 110% of the previous duty cycle, greater 120% of the previous duty cycle, and greater 130% of the previous duty cycle.

20. The aerosol provision system of claim 1, wherein the aerosol generator is an electric heater.

21. The aerosol provision system of claim 1, further comprising the aerosol generating material.

22. The aerosol provision system of claim 1, wherein the aerosol provision system comprises a control unit part and a consumable part detachably couplable to the control unit part, and wherein the control unit part comprises the control circuitry and the consumable part comprises the aerosol generator.

23. A control unit for an aerosol provision system comprising:
a power source;
control circuitry configured to cause the power source pulses of electrical current having a duty cycle to an aerosol generator in use so as to maintain a substantially constant average power,
wherein the duty cycle is dependent on the temperature of the aerosol generator; and
wherein the control circuitry is configured to determine a voltage supplied by the power source.

24. A control unit for an aerosol provision system comprising:
a power source;
control circuitry configured to cause the power source to supply electrical current having a duty cycle to an aerosol generator in use so as to maintain a substantially constant average power, wherein the duty cycle is dependent on the temperature of the aerosol generator;
wherein the control circuitry is configured to determine the duty cycle and to compare the duty cycle with a first duty cycle threshold dependent on a previous duty cycle.

25. Aerosol provision means comprising:
power source means;
control means configured to cause the power source means to supply electrical current having a duty cycle to aerosol generating means so as to maintain a substantially constant average power, wherein the duty cycle is dependent on the temperature of the aerosol generating means; and
wherein the control means is configured to determine a voltage supplied by the power source means.

26. Aerosol provision means comprising:
power source means;
control means configured to cause the power source means to supply electrical current having a duty cycle to aerosol generating means so as to maintain a substantially constant average power, wherein the duty cycle is dependent on the temperature of the aerosol generating means; and
wherein the control means is configured to determine the duty cycle and to compare the duty cycle with a first duty cycle threshold dependent on a previous duty cycle.

27. A method of operating an aerosol provision system comprising control circuitry and a power source, wherein the control circuitry performs the method of:
determining a voltage supplied by the power source; and
causing the power source to supply electrical current having a duty cycle to an aerosol generator so as to maintain a substantially constant average power, wherein the duty cycle is dependent on the temperature of the aerosol generator.

28. A method of operating an aerosol provision system comprising control circuitry and a power source, wherein the control circuitry performs the method of:
causing the power source to supply electrical current having a duty cycle to an aerosol generator so as to maintain a substantially constant average power, wherein the duty cycle is dependent on the temperature of the aerosol generator; and
comparing the duty cycle with a first duty cycle threshold dependent on a previous duty cycle.
